(19) United States
(12) Patent Application Publication
(10) Pub. No.: US 2017/0266247 A1
(43) Pub. Date: Sep. 21, 2017

(54) A NOVEL SYNERGISTIC HERBAL FORMULATION FOR DEGENERATIVE DISEASE CARDIOVASCULAR DISEASES, AUTOIMMUNE, INFLAMMATORY AND METABOLISM DISORDER AND THE PROCESS OF PREPARING THE SAME

(71) Applicant: Akhawri Shankar, Jharkhand (IN)

(72) Inventor: Akhawri Shankar, Jharkhand (IN)

(21) Appl. No.: 15/316,524

(22) PCT Filed: Jan. 4, 2016
(86) PCT No.: PCT/IN2016/000001

§ 371 Date: Dec. 6, 2016

(30) Foreign Application Priority Data
Nov. 9, 2015 (IN) 1138/KOL/2015

(51) Int. Cl. A61K 36/48; A61K 36/486

(52) U.S. Cl. CPC A61K 36/48; A61K 36/486; A61K 2236/17

(57) ABSTRACT

The present invention relates to a novel synergistic herbal formulation for degenerative disease, cardiovascular diseases, autoimmune, inflammatory and infectious diseases and metabolism disorder etc. of man, animal and plants More particularly, the present invention relates to the herbal formulation for degenerative disease, cardiovascular diseases, autoimmune, inflammatory and infectious diseases etc which is prepared by using preparation of pongamia pinnata, Dalbergia sissoo (shishu) and Syzygium aromaticum (clove) which is used as directly and neuro pathways. Moreover this invention relates to the process for the preparation of the above composition.

### CROSS-REFERENCE TO RELATED APPLICATIONS

This application claims the priority of Indian non provisional patent application no: 1138/KOL/2015 filed on 9 Nov. 2015 with the title “A NOVEL SYNERGISTIC HERBAL FORMULATION FOR DEGENERATIVE DISEASE CARDIOVASCULAR DISEASES, AUTOIMMUNE, INFLAMMATORY AND METABOLISM DISORDER AND THE PROCESS OF PREPARING THE SAME” and the content of which is incorporated hereby by reference in its entirely.

### BACKGROUND

Technical Field

The embodiment herein of the invention relates to a novel synergistic herbal formulation for degenerative disease, cardiovascular diseases, autoimmune, inflammatory and infectious diseases and metabolism disorder etc. of man, animal and plants More particularly, the embodiment herein of the invention relates to the herbal formulation for degenerative disease, cardiovascular diseases, autoimmune, inflammatory and infectious diseases etc which is prepared by using preparation of pongamia pinnata, Dalbergia sissoo (shishu) and Syzygium aromaticum (clove) which is used as directly and neuro pathways. Moreover this embodiment herein of the invention relates to the process for the preparation of the above composition.

Background of the Related Prior Art

Biodiversity forms the basis for a healthy environment and source of economic and food security for future generation. Every organism and its variations are unique and have its own role to play a particular ecological niche. At least 40% of the world economy and 80% of the need of the poor are dependent on the biological material. The traditional system serves as a cure to the 80% of the total population.

About 80% of the world's population relies solely or largely on traditional remedies for their healthcare needs. Today, about 70,000 to 80,000 plant species are used for medicinal or aromatic purposes globally. India with its ecological, geographical and climatic diversities is perhaps the richest nation with a vast herbal medicinal wealth (About 15000-20000 plants have good medicinal value). In india the therapeutic use of herbs dates back to the vedic period. The Rigveda has documented about 67 medicinal plants, Yajurveda 81 species and Atharvaveda 290 species.

The local environment specially the range land flora governed the needs of the resource poor farmers. So the local therapies especially the plant based therapies varied considerably from one region to other. Many of the present day remedies were borrowed from folk medicine, so there is paramount need to document and validate this indigenous system of treatment and collate with the scientifically validated knowledge, so as to have better understanding the rationale behind such practices.

Cloves are used in Indian Ayurvedic medicine, Chinese medicine, and western herbalism and dentistry where the essential oil is used as an anodyne (painkiller) for dental emergencies. Cloves are the aromatic flower buds of a tree in the family Myrtaceae, Syzygium aromaticum. Cloves are used as a carminative, to increase hydrochloric acid in the stomach and to improve peristalsis. Cloves are also said to be a natural anthelmintic. The essential oil is used in aromatherapy when stimulation and warming are needed, especially for digestive problems. Topical application over the stomach or abdomen are said to warm the digestive tract. Applied to a cavity in a decayed tooth, it also relieves toothache.

In Chinese medicine, cloves or ding xiang are considered acrid, warm, and aromatic, entering the kidney, spleen and stomach meridians, and are notable in their ability to warm the middle, to treat hiccough and to fortify the kidney yang. Because the herb is so warming, it is contraindicated in any persons with fire symptoms and according to classical sources should not be used for anything except cold from yang deficiency. As such, it is used in formulas for impotence or clear vaginal discharge from yang deficiency, for morning sickness together with ginseng and patchouli, or for vomiting and diarrhea due to spleen and stomach coldness.

Cloves may be used internally as a tea and topically as an oil for hypotonic muscles, including for multiple sclerosis. This is also found in Tibetan medicine Some recommend avoiding more than occasional use of cloves internally in the presence of pitta inflammation such as is found in acute flares of autoimmune diseases Cloves (Syzygium Aromaticum), many medicinal uses have been most famously applied to toothache, and for mouth and throat inflammation. The dove has been used in India and China, for over 2,000 years, as a spice to check both tooth decay and counter halitosis that is bad breath. In Persia and China, it was considered to have aphrodisiac properties. Cloves have historically been used in Indian cuisine (both North Indian and South Indian). In the north Indian cuisine, it is used in almost every sauce or side dish made, mostly ground up along with other spices. More than just a counterirritant though, the German Commission E Monographs list cloves as having antiseptic, antibacterial, antifungal and antiviral properties One of the main constituents of clove oil (eugenol) exhibits broad antimicrobial activities against both Gram-positive, Gram-negative and acid-fact bacteria, as well as fungi Cloves are well known also for their antiemetic (relieves nausea and vomiting) and carminative properties.

The invention cited in the document WO2012067592, a ready to use clove oil formulation for anesthesia for aquatic animals comprises an effective amount of a preparation comprising clove oil, polyethylene glycol 400, propylene glycol, ethyl alcohol, and surfactant to anesthesize, reduce mobility and reduce the rate of metabolism, wherein said preparation can dissolve and distribute well in water (i.e. presenting no floating clove oil on the water surface). The formulation also comprises of other pharmaceutical aids e.g. anti-oxidative agents, chelating agents, preservative agents and coloring agents. The anti-oxidative agents are phenolic antioxidants as well as reducing agents (e.g. ascorbic acid, ascorbyl palmitate, etc.). Examples of the chelating agent are citric acid, disodium ethylenediaminetetraacetic acid (Disodium EDTA). Examples of the preservative agents are parabens, benzyl alcohol, chlorobutanol, thiomersal, nitromersol, benzalkonium chloride, cetylpyridinium chloride. The coloring agents are water-soluble coloring agents (dyes).

The document CN104248654 describes an application of a Pongamia pinnata general flavone extract or karanjin in the preparation of influenza virus treatment and/or prevention drugs. The Pongamia pinnata general flavone extract is extracted from stem branches and/or leaves of Pongamia pinnata, contains 50-70 wt % of general flavones, and also contains karanjin; the content of the general flavone is determined through an ultraviolet spectrophotometric method with karanjin as a reference substance; and the content of karanjin is 5-10 wt %.

The document US2007254018 describes a topical medicant for fishes, amphibians, reptiles, and aquatic macroinvertebrates applied in a site-specific manner for the localized treatment of skin wounds, lesions and diseases. The medicant comprises a hydrophilic carrier with an antimicrobial therapeutant formulated as a water-soluble resistant, hydrated gel or dry powder that hydrates and gels on contact with a moistened integument area.

This invention of CN101259167 relates to an anesthesia method for carp, which belongs to the technical field of aquiculture. The invention is characterized in that the water of 99.98 to 99.99 weight percentage and the clove oil of 0.01 to 0.02 weight percentage are added in to a container to prepare anesthesia solution, and the carp is put into the container to be anesthetized. After being anesthetized for 2 to 5 minutes, the carp is found lying on side in the solution. And then, the carp is put into the waters of a pond. After 5 to 8 minutes, the crap can swim as usual. The anesthesia method of the invention adopts the clove oil that is commercially available and has good anesthesia effect. The clove oil can be used for soothing toothache of human. So the clove oil is safe to anesthetize the carp and does not affect edibleness. In addition, the clove oil has the advantages of low cost and wide application range.

A herbal composition as stated in document CN101238801 the field of aquaculture technology relates to a method of anesthesia for tilapia. The invention is characterized in that, infuse warm water into the container, the water temperature is 18 DEG C.-30 DEG C.; infuse narcotic clove oil into container to prepare for the clove oil solution; put the tilapia into the clove oil solution, the anesthesia time is: 2-5 minutes, then move the anaesthetic anesthesia into the pool for recovery of 3-8 minutes, the tilapia is recovered to swim normally. The invention applies the clove oil on the market as the anesthesia to anaesthetize the tilapia, the anesthesia effect is good; the clove oil can be used as tooth pain killer for human, so the anaesthetic anesthesia is safe to eat; the price is low, and the applied range is wide.

The other document XP002652780, DONG Z and ZHU Z, Vol:2008 describes anesthetizing carp comprises adding water and clove oil to the container for preparing aesthetic solution, putting carp into aesthetic solution and anesthetizing, and moving the carp which has been anesthetized to the water tank

F. C. Tuteja et al (2011) says Survey was carried out in thickly camel populated areas in semi arid climatic conditions (Udaipur and Hanumangarh districts) and in arid climatic conditions (Bikaner and Jaisalmer districts) of Rajasthan state of India, to investigate the ethno veterinary methods practiced by the camel owners for the treatment and control of various ailments affecting camels. During a 3 year period (July 2007-June 2010) a total of 410 camel owners including the persons recommending such practices to them were personally contacted and interviewed. These owners were rearing from a minimum of one camel to a maximum of 160 camels. Information concerning the ethno-veterinary practices for the treatment of common disorders of skin (maggot wound, nasal wound, saddle gall, udder lesion, naval infection, mange, thikria i.e. skin candidiasis and other skin infections), digestive tract (anorexia, indigestion, impaction, colic, constipation, diarrhoea, tympany), musculoskeletal system (rheumatism, muscular weakness, kumree i.e. shivering of hind limbs, enhance healing of fracture and deep wounds) and other routine systemic disorders encountered by the farmers for fever, cough, nasal discharge, pneumonia, haemorrhages, mastitis, actinobacillosis, retention of placenta, helminthiasis, ectoparasites, eye infection, sun stroke, utrine prolapse and zeharbad was collected through personal discussions and interviews. Traditional inputs utilised by the camel owners included various plant products, animal products, other natural products and a very few synthetic products. Method of preparation and applications of traditional drugs varied as per the ailment to be treated. Firing remained the last resort by the camel owner, if the animal did not respond to any kind of treatment. Preventive practices adopted by the camel owners included; breeding studs were given red alum, jaggery and mustard oil during breeding season to avoid fatigue and increase libido. Exercise in fresh lactating camels to avoid mastitis and smoke in the sitting area to avoid insect and flies. This traditional knowledge was collated with scientifically validated knowledge concerning any ingredients or active constituents of the ingredients. This collation revealed scientific relevance behind many traditional practices. Predominance of use of a particular therapy varied mainly as per the availability of the product in a particular area. Frequency of use can be correlated with either the effectiveness of a particular therapy or effective transmission of therapeutic knowledge among the farmers.

K. N. GAIKWAD and M. V. MALI (2012) states that a fairly good number of exotic tree species were reported in the area due to their introduction in the past 2-3 decades by Municipal Corporation, social forestry department, nursery and industry owners etc. On the other hand many indigenous trees have been depleted due to increasing urbanization. In our opinion, this is the main reason to the increased number of exotic taxa. Most of the exotic trees have been acclimatized well and increased the greenery and beauty of the city. It is observed that some of the trees have provided shelter for birds and insect pollinators while few produce allergic pollens. We do not know the effects of the new introductions over native vegetation yet.

This invention of the document 1401/CHE/2008 relates to a synergistic polyherbal composition for the treatment of bronchial asthma, said composition comprising: Achyranthes aspera: 15.5%-17.5% by wt % Woodfordia furiticosa 11.5%-13.6% by wt % Solanum xanthocarpum: 0%-13.6% by wt % Justicia adhatoda: 11.5%-13.6% by wt % Acacia arabica: 11.5%-13.6% by wt % Zingiber officinalis: 3.6%-4.94% by wt % Syzygium aromaticum bud: 3.6%-4.94% by wt % Curcuma longa: 3.6%-4.94% by wt % Holarrhena antidysentercia: 3.6%-4.94% by wt % Enicostemma littorale: 2.22-3.3% by wt % Calotropis procera: 2.22%-3.3% by wt % Piper longum: 2.22%-3.3% by wt % Piper nigrum: 8.2%-7.8% by wt % Elettaaria Cardamomum: 0.06%-0.19% by wt %.

The invention relates to the use of compositions comprising herbal preparations for the treatment and prevention of plant diseases caused by pests such as nematodes, fungi and insects. Said compositions comprise a preparation or extract of two or more plants selected from Pimpinella anisum, Peganum harmala, Myristica fragrans, Syzygium aromaticum and Rhazya stricta. Also provided are methods for the treatment or prevention of disease in plants which comprise applying said compositions; seeds, plant growth media, food wrapping and pesticide formulations comprising said compositions; and methods for the prevention of termite damage to buildings which comprise applying said compositions to buildings or to building materials as stated in the document 1633/MUMNP/2010.

The invention stated in the document 1840/DEL/2010, a pleasant-tasting, substantially oral non foaming anti-precancerous and anti-microbial toothpaste composition is effective in oral leukoplakia, oral lichen planus, submucous fibrosis etc and eliminating the bacteria and other oral microflora responsible for the production of plaque, periodontitis, gingivitis, gum disease and bad breath. The composition consists of a unique blend of curcumin, curry leaves, allium sativum and tea tree oil, Elettaria cardamomum, Syzygium aromaticum, Foenieulum vulgare, silica, Glycerol, sodium carboxymethyl-cellulose, sodium saccharin and Propyl Paraben.

The document 2027/DEL/2011 states discloses a novel herbal formulation which is effective in preventing and curing dandruff. The herbal formulation essentially comprises peel of citrus (Citrus sp.), roots of licorice (Glycyrrhiza glabra) triphala [equal mixture of fruits of amla (Embelica officinalis), bahera (Terminalia bellerica) and harad (T. chebula)], neem leaves (Azadirachta indica), green tea leaves (Camelia sinensis), eucalyptus leaves (Eucalyptus sp.), henna leaves (Lawsonia inermis) and clove oil (Syzygium aromaticum). The herbal formulation has fungicidal action towards dandruff causing fungus. The herbal formulation may be in different forms for application such as aqueous extract or can be formulated in a shampoo for end use.

The document 2932/DEL/2011 discloses a polyherbal formulation for healing and caring that is cost effective, friendly to the skin, and a method of preparation thereof. The formulation comprising of Curcuma longa (Turmeric) extract in Ricinus communis (Castor) oil, Azadirachta indica (Neemi) oil with essential oils of Boswellia serrata (Salai Guggal), Cymbopogon nardus (Citronella, among others), and Syzygium aromaticum (Clove).

The document 4613/CHE/2011 disclosed herein is a synergistic supplemented herbal composition and process for preparing the same. The said herbal composition comprises 0.1-5% extract of herbs in powder form namely Achyranthes aspera Woodfordia furiticosa Solanum xanthocarpum Justicia adhatoda Acacia arabica Zingiber officinalis Syzygium aromaticum bud Sygygium aromaticum oil Curcuma longa Holarrhena antidysentercia Enicostemma littorale Calotropis procera Piper longum Piper nigrum Elettaaria Cardamomum and 95-99% edible refined salt used for treatment of allergy related illnesses. The process for preparing said composition comprises extracting 0.1 to 5% of herbs with hydro alcoholic solvent spray drying the said extract into powder form and thereafter blending it with 95%-99.9% refined salt to get the required composition.

The invention in the document 808/MUM/2013 disclosed herein is a herbal composition comprising extracts of herbs selected from Hypericum mysorense, Withania somnifera, Phyllanthus niruri, Holoptelia integrifolia, Acacia Catechu, Vetiveria zizanioides, Syzygium aromaticum, Terminalia chebula, Azadirachta indica Linn, Glycyrrhiza glabra, Bacopa monnieri and Andrographis paniculata along with pharmaceutical acceptable excipients, useful for the treatment of symptoms associated with Herpes simplex virus, Human papilloma virus and other viral infections.

The document 1734/DEL/2013 relates to an herbal synergistic anti-mite formulation. The composition comprises the extracts of Curcuma longa, Artemisia pallens, Chromolaena odorata and Syzygium aromaticum in Azadirachta indica seed oil. The process comprises the preparation of the extracts of the above herbs and the composition is in topical dosage form selected from the group of oil or ointment or cream.

The invention in the document 2424/DEL/2013 stimulating Chewable Processed Compound is a novel product which will fulfil and replace chewing habit of individuals in replacement of harmful chewing products like Gutkha, Panmasala others any kind of betel nuts etc. This product uses Chowahara as base product and uses common herbs viz Saunf (Fennel); Foeniculum vulgare, Cardamom seeds, Clove (seed of Syzygium aromaticum); Jaiphal (Nutmeg): seeds of Myristica fragrans, Pudhina, Tulsi, Kewara and Saffron as Stimulating and flavouring ingredients. The base material is roasted at an optimum temperature for an optimum time and then the herbs are sprinkled in form of powder which act as stimulating materials for the brain and will deaddict a person of Tobacco. The herbs have been tried in various ratio and an ideal ratio have been developed which de-addicts the Tobacco habit. The herb will be able to act as a de-addiction chewing material for Tobacco users. It will also be useful as mouth freshener and as digestive agent and also for chewing it at time of deep stress and depression. It will thus act as antidepressant also without having any side effects.

Therefore, attention is being paid to find alternative approaches. This has prompted interest in finding new, effective, safe herbal compositions comprising plant extracts to treat cardiovascular diseases, autoimmune, inflammatory, degenerative changes and infectious diseases etc. The extracts of various plants that are native to the tropics and subtropics are being tested and some have demonstrated some activity against various tick species. A number of herbs have been found to be effective in treating degenerative disease, cardiovascular diseases, autoimmune, inflammatory and infectious diseases etc. While research in the field of herbal medicines has increased, much remains to be learned about the effectiveness of such herbal remedies for rheumatism, skin diseases and scabies infections etc.

### OBJECT OF THE EMBODIMENTS

Accordingly, there still exists an ongoing need for developing new synergistic herbal extract combination which are safe, and effective against microbial infections yet devoid of side effects so as to rejuvenate the general health and immunity of the human beings. The present invention satisfies these needs, as well as others, and generally overcomes the deficiencies found in the background art.

Another object of the present invention is to provide a novel synergistic herbal formulation for degenerative disease, cardiovascular diseases, autoimmune, inflammatory and infectious, degenerative disease and metabolism diseases.

Still other object of the present invention to provide a formulation which comprises using the preparation of the various part of pongamia pinnata and Dalbergia sissoo (shishu) and Syzygium aromaticum (clove).

Another object of the present invention is to provide the novel herbal composition which is safe, effective and devoid of side effects.

Another object of the present invention is to provide the novel herbal composition which is having economic significance in comparison to the commonly available allopathic and synthetic drugs.

### SUMMARY

Furthermore, another object of this invention relates to the process for the preparation of the above composition by using the above composition and composition comprises using the preparation of the various part of pongamia pinnata which is also acts for remedy of cholera and E. coli and Dalbergia sissoo (shishu) and part of Syzygium aromaticum (clove).

Another object of the present invention is to provide the herbal composition wherein Syzygium aromaticum (clove) is used in 2% of the total wt..

Another object of the present invention is to provide the herbal composition and medicament thereof of the present invention is simple, natural, attractive and convenient to use.

Another object of the present invention is to provide rosewood which stimulates new cell growth, regenerate tissues.

Another object of the present invention is the manufacturing method for herbal medicine is simple and the effect is excellent; the price is low. The herbal composition and medicament thereof is useful in the treatment of human being.

Novelty of this instant invention lies on the composition of the ingredients of above herbal composition in a particular ratio and their process of preparing the composition.

The herbal composition of the impugned invention is used for the treatment of cardiovascular, auto immune, inflammatory, infectious and degenerative changes diseases disorder, slipped disc, spondylitis, frozen shoulder osteoporosis, peri capsulitis, ankylosing spondylitis, diabetes, scoliosis, osteoarthritis, arthritis, rheutism, sleeping sickness, mental disorder, asthma, SLE, throat infections, pharyngitis, cerebral defused atrophy, bales palsy, paralysis, coronary arterial diseases, cholesterol anemia Blood pressure amia, migraine, fatty lever, cystic problem, sinusitis, cancer chicken gunia, dengue bird flu, burns, capsular muscle and other related diseases of metabolism disorder of a man, animal and plants. The natural additive can be used to make the composition in liquid or solid or semisolid form.

The use of the aforesaid plant molecules/cells in the neurogenesis pathways and of metabolites and breakdown products formed in the metabolism and breakdown pathways related to various molecules in the neurogenesis pathway as treatments. Both alone and various combinations with themselves and in combination with the treatment to repair the metabolism by such techniques as the use of the chelating agents and by corrections in various biochemical pathways that lead to repair damage containing compound, protein the body uses to rid itself of the protein.

Still another object of the present invention is to provide a process which comprises, the ingredients of pongammia pinnata(liquid form) and Dalbergia sissoo(shishu) and Syzygium aromaticum (clove) is used as a whole or the portion of the tree as required in a certain percentage. The ingredients are as per volume/weight are roasted separately in sun rays for a 2 to 3 days. After that the mixture is prepared by volume by weight are heated till boiling temperature for a certain time. The extract formed is then cooled till it comes in room temperature. This extract is then processed of purification so that no solid and malformation comes out. The processed liquid is then kept in airtight container.

The way of external administration of the above composition for the various treatment are depending the disease.

### BRIEF DESCRIPTION OF THE DRAWINGS

The efficacy of the present invention lies in the formulation of the composition of the ingredients in a particular ratio which is highly significant as shown by the examples herein below.

A group of six patients with chronic degenerative diseases were selected on the basis of diseases were excluded the medicine were applied on the patient, the outcome measures were remarkable. The incidence and severity if treatment related adverse effects were also monitored.

The effects of the medicine infection on human metabolism were examine in diseases restricted to the loss of neural function or cardio synaptic changes.

Patient 1 suffered from coronary arterial disease CAD that was treated with this medicine. The patient was examined at Spanda Hospital Hyderabad to monitor the effects of the medication and disease. The following results were observed.

TABLE 1

| | |
|---|---|
| Mitral valve | Normal |
| Tricuspid vlave | Normal |
| Pulmonary valve | Normal |
| Aorta valave | Normal |
| Aorta | 4 cm |
| Pulmonary artery | Normal |
| Pulmonary veins | Normal |
| Left atrium | 4 cm |
| Left ventricle | Normal. |
| LVIDS | 3.5 CM |
| LVIDD | 5.5 CM |
| LVPW | 1/1.8 CM |
| LVEF | 67% |
| FS | 38% |
| LEFT VENTRICLE POST WALL | Normal |
| Interventricular septum(ivs) | 1/1.8 cm |
| Interarterial septum(ivs) | Normal |
| Right atrium | Normal |
| Right ventricle | 1.4 cms |
| Pericardium | Normal |
| Great artery relation | Normal |
| Doppler study | Normal |
| Colour Doppler | Normal |
| Tricuspid valve flow | Normal |
| Aortic valve flow | Normal |
| Colour flow mapping | Normal |

MR N+/+/+/+/+/+/+/+/+/
TR N+/+/+/+/+/+/+/+/+/
AR N+/+/+/+/+/+/+/+/+/
Normal valves/blood flow, normal RWMA/LVH Good V function.

Patient 2 presented with chronic pain in his left knee, which severely restricted mobility and was non responsive to extensive pain management. On examination the patient's knee exhibited degenerative changes with reduction in space owing to slight medical swelling and soft tissue damage (FIG. 1A). The patient received the study medication for 90 days following the appearance of degenerative changes in the knee .post treatment x-rays of the knee are presented in FIG. 1B. No abnormalities or degenerative changes were observed.

Patient 3 exhibited bilateral reduced space with degenerative changes in both knees. (FIG. 2A) The medication was investigated in both knees for six months with four administrations per month. The x-rays revealed remarkable changes in both knees (FIG. 2B)

Patient 4 presented with carcinoma of the prostate gland that had metastasized to skeleton (FIG. 3A) specific antigen (PSA) score was 22.70; the prostate biopsy Gleason score was 3+3=6.00 The study medication was investigated in the 5^{th} vertebra. The medication was administered for three months after the initial bone isotope scan and the follow up scan revealed decreased intensity in this vertebra (FIG. 3B). After treatment the total PSA score had fallen to 0.144 whereas the free PSA score was 0.11 in addition, both pre-treatment and post treatment blood counts was performed. The pre-treatment monocyte and basophil counts were 8.2-9.2 and 0.4 respectively. by contrast neither monocytes nor basophils were detected in the post treatment blood screen.

Patient 5 presented with inflammation and degenerative changes in sacroiliac joint (FIG. 4A) DNA analysis confirmed the presence of the HLA B27 human leukocyte antigen and ankylosing spondylitis. Despite the use of pain medication, he was unable to walk or sleep and remained in extreme pain. The medication was administered to this patient with ankylosing spondylitis. The medication was trans infected for four months i.e. ninety days after which the stiffness in his sacrolliac joint was eliminated. No pain or bony lesions were observed in the sacroiliac joint (FIG. 4B).

Patient 6 presented with proximal distal weakness in both upper and lower calves for the last four years. The weakness was progressive and symmetric with atrophy in both calves. Reflexes were absent and without sensory symptoms, which suggested limb grid muscular dystrophy. Review of the pathology revealed congenital myopathy with protein aggravates. The pre treatment aldoses level was 8 U/L and the creatinine phosphokinase (CPK) level was 688 U/L. This patient received the study medication for six to seven months, after which her aldoses level was reduced to 4.1 U/L, while the CPK level had fallen to 275 U/L

Different combinations have been tried which are illustrated by the following examples.

The novel features that are considered characteristic of the present invention are set forth with particularity in the appended claims. The invention itself, however, both as to its organization and its method of operation, together with additional objects and advantages thereof, will best be understood from the following description of certain specific embodiments, when read in connection with the accompanying drawings, in which:

FIG. 1 illustrates: Pre-treatment (A) and post-treatment (B) x-rays showing the left knee of patient of the present invention;

FIG. 2 illustrates Pre-treatment (A) and post-treatment (B) x-rays showing both knees of patients 3 of the present invention;

FIG. 3 illustrates pre-treatment (A) and post treatment (B) whole body scan of the present invention;

FIG. 4 illustrates pre-treatment (A) and post-treatment (B) x-rays showing the sacroiliac region of patient of the present invention;

FIGS. 5A and 5B illustrates pre-treatment (A) and post treatment (B) of patient of the present invention;

FIGS. 6A and 6B illustrates pre-treatment (A) and post-treatment (B) of patient of the present invention;

FIGS. 7A and 7B illustrates pre-treatment (A) and post-treatment (B) of patient of the present invention;

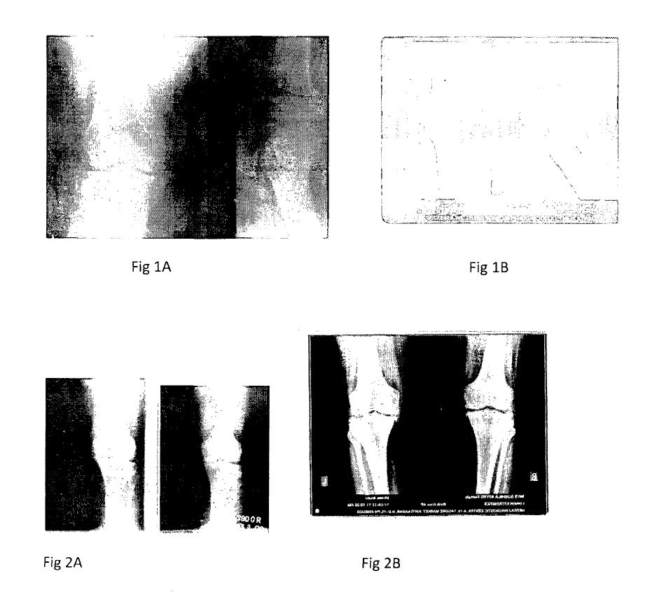

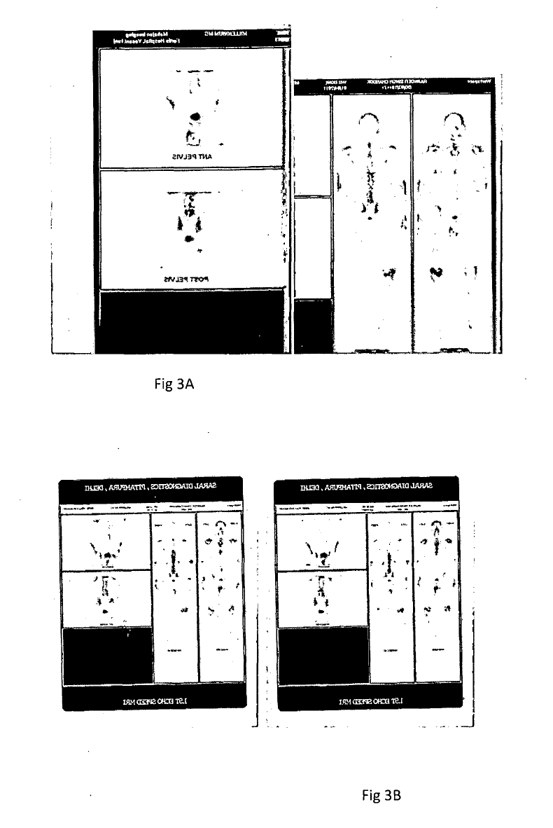

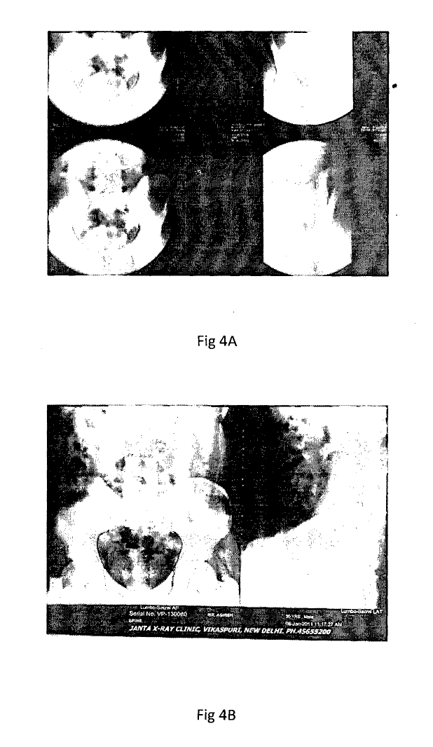

### EXAMPLE 1

A group of seven patients with chronic degenerative disease, infectious disease, cardiovascular disease auto immune diseases and inflammatory disease who were treated with the composition and result are as follows:

### Patients 1

Mr Rikhab jain 35 yrs a private employee and a resident of Coimbatore had cervical spondylitis. For a few years, everything appeared normal. Then the condition got aggravated. A series of consultations for serious lengths of time followed with general practitioners, physiotherapists homeopathic doctors and so on. Soft mattresses, cushioned pillows and cervical collars were tried. You name the treatment, he had undergone it, but no avail. He had only two applications of the medicine and cured completely.

### Patients 2

Dr Meena azad, 40 years with a frozen shoulder problem. The frozen shoulder is an auto immune per capsulitis of the shoulder joint. It leads to severe pain restrictions in movements. The disease becomes chronic; it may take 9 months to 2 year to get relief. Not usually, the other shoulder becomes effected thereafter. Usually, the frozen shoulder or pericapsulitis symptoms disappear after 6-8 months. But, in the case of Dr Meena Azad they had persisted for ten years. The shoulder had limitations of movement to a great degree; combing hair was very difficult and painful. She had only two applications of this treatment. Pain disappeared leading to free movement.

### Patients 3

Thirty-six year old Madhukant Pathak is suffering from slipped disc and that put him out of action. Slipped disc is a common problem. It occurs while lifting weight not necessarily heavy action ones. This results in pain and restricted movement in the spine. The spinal cords may get pressed and this may result in motor and sensory problem in the lower limbs and the neurological signs like increased tendon reflexes up going may occur. The MRI of spinal cord vividly showed the effect of the pressure on the cord. He was treated with composition of the impugned invention and free from disease.

### Patients 4

Mr Danish has been suffering from scoliosis of the spine. Scoliosis is gene defect disease (NF1 gene). In scoliosis the spine become curved. A spine with scoliosis has abnormal curves with rotational deformity. This means that the spine on its axis like a corkscrew. His spine is not straight but tortuous like serpent. He was treated with composition of the impugned invention and free from disease.

### Patients 5

Fifty-two years old man had been suffering from asthma is a chronic lung disease, caused by inflammation of lower airways with episodes of airflow obstruction. Asthma is characterized by recurrent breathing problems caused by airways inflammation. The symptoms of asthma vary among people and include wheezing, shortness of breathe and tightness of the chest. He was treated with composition of the impugned invention and free from disease.

### Patients 6

Mr R. K. Gupta, age 45 years had been suffering from ankylosing spondylitis since 1985. Ankylosing spondylitis is a chronic condition in which the spine becomes rigid due to fibrous growth around the spine and also called “Bamboo spine” (HLA B27 gene defect). Reduced movement of the joints neck and back pain in joints, hip, ankle, feet as the case may be fixed spine with forward curvature are the main symptoms. He was treated with composition of the impugned invention and free from disease.

### Patients 7

Mr Pratap Chandra aged 48 yrs had been suffering from diabetes mellitus for few years. He had to take insulin regularly before food He had abscess and other complications. The super level; 343 mg/dl fasting, 252 mg/dl after taking insulin after food of two hours. The medicine was applied in the spine on two days one time each. A checkup was done. The sugar level was 200 mg/dl fasting and 121 mg/dl after taking insulin after food. After seven days, the medicine was applied again in spine. The glucose plasma came down to 106 mg/dl. He is fit and now he is taking just a single dose of medicine at night. He was treated with composition of the impugned invention and free from disease.

### EXAMPLE 2

Three patients with chronic infectious with degenerative diseases are selected for the treatment by using the composition of the impugned invention.

### Patient 1

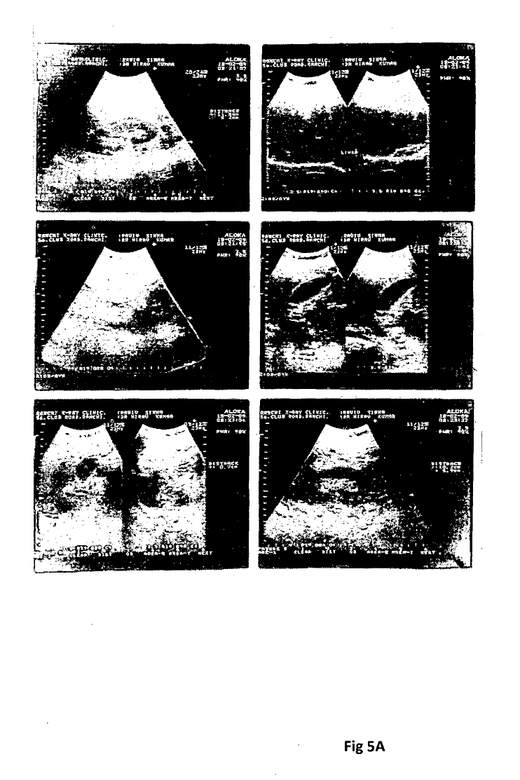

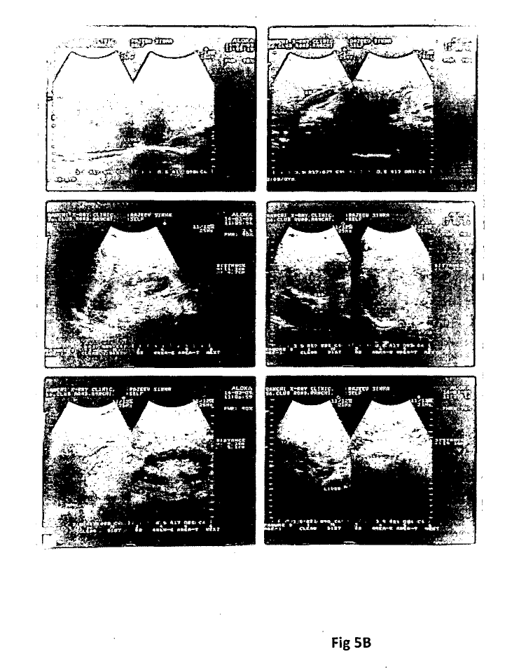

Suffered from the cystol and fatty lever gr III FIG. 5A. (USG). The patient has been treated with this medicine. The patient was examined free from diseases. The result were observed remarkable changes FIG. 5B (USG).

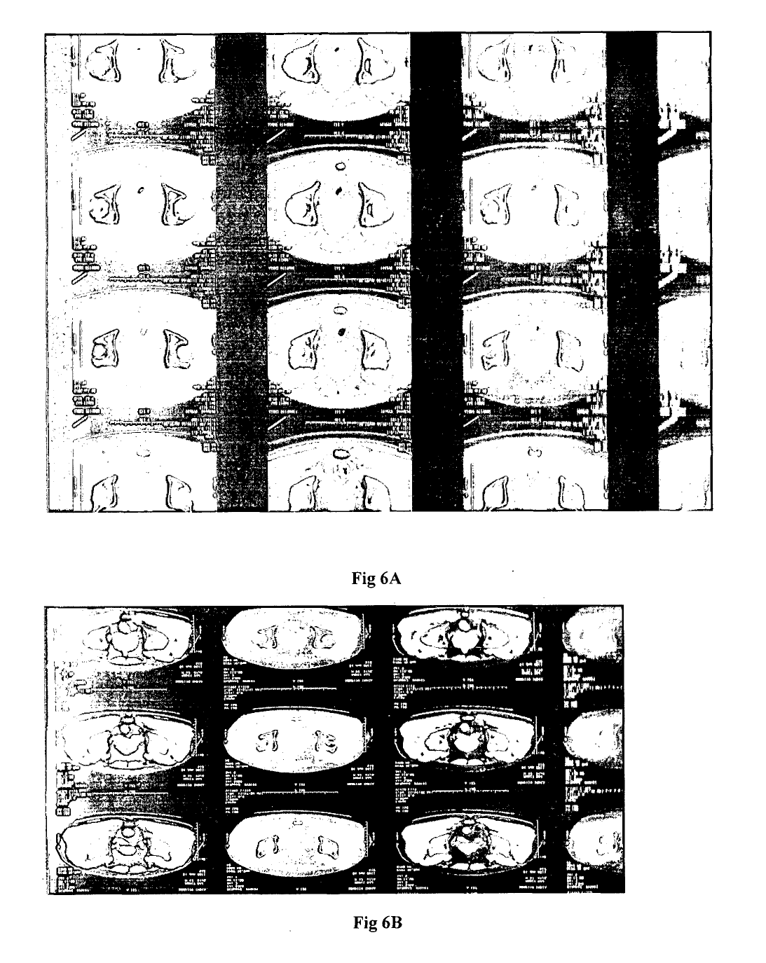

Patient 2 exhibited reduced sacroiliac joint with distorted, degeneration & infection (FIG. 6A). The medicine was investigated in sacroiliac joint with eighteen administration within three month MM revealed remarkable changes FIG. 6B.

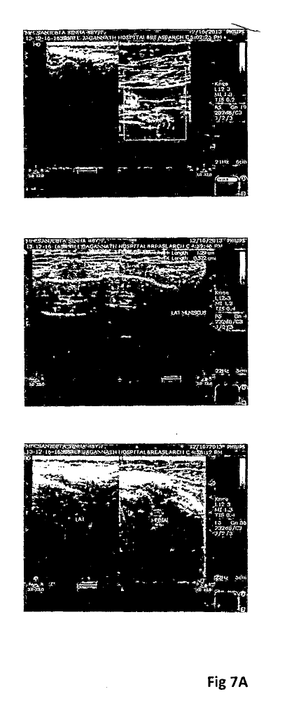

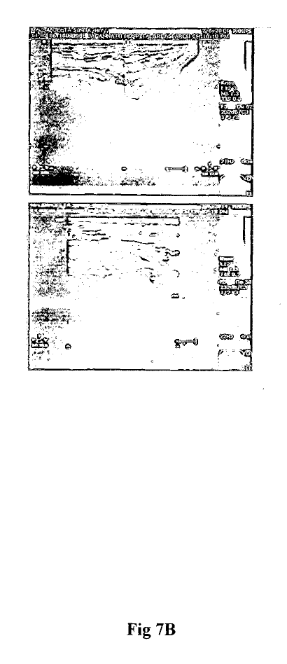

Patient 3ie 48 yrs female was unable to walk always pain in right kee swelling USG done in right keen ameniscal cyst bearing the size of 1.29×0.52 cms. FIG. 7A treated by this medicine in three months eighteen application a miraculous report seen in the USG .no cyst seen in the FIG. 7B.

### Advantages of the Invention

1. The present invention provides a novel synergistic herbal extract composition comprising therapeutically effective amounts of plant extracts.
2. The present invention provide use of the novel herbal composition of the present invention for treating or preventing degenerative disease, cardiovascular diseases, autoimmune, inflammatory and infectious diseases etc in a subject in need thereof
3. The present invention provides a process for the preparation of novel herbal composition of the present invention.
4. The present invention provides the novel herbal composition which is safe, effective and devoid of side effects.
5. The present invention provides the novel herbal composition which is having economic significance in comparison to the commonly available allopathic and synthetic drugs.

The present invention as described above, it is to be understood that this invention is not limited to particular methodologies and materials described, as these may vary as per the person skilled in the art. It is also to be understood that the terminology used in the description is for the purpose of describing the particular embodiments only, and is not intended to limit the scope of the present invention.

Therefore, the foregoing is considered as illustrative only of the principles of the invention. Further, since numerous modifications and changes will readily occur to those skilled in the art, it is not desired to limit the invention to the exact formulation as described, and accordingly, all suitable modifications and equivalents may be resorted to, falling within the scope of the invention.

What is claimed is:

1) A novel synergistic herbal formulation which comprises an effective amount of mixture of pongamia pinnata (liquid form) and Dalbergia sissoo (shishu) and Syzygium aromaticum (clove) whole or part of tree as required.

2) The novel synergistic herbal formulation as claimed in claim 1 wherein the above mixture contains rosewood such as herein described.

3) The novel synergistic herbal formulation which comprises an effective amount of various part of the above said trees.

4) The novel synergistic herbal formulation as claimed in claim 1 wherein all the ingredients in mixture are roasted.

5) The novel synergistic herbal formulation as claimed in claim 1 wherein the Syzygium aromaticum (clove) content is 2%.

6) The novel synergistic herbal formulation as claimed in claim 1 wherein the formulation is safe, effective and devoid of side effects.

7) The novel synergistic herbal formulation as claimed in claim 1 wherein the natural additive can be used to make in the liquid or solid or semisolid form.

8) A process for preparing the novel synergistic herbal formulation as claimed in claim 1 which comprises;
a) all the ingredient of mixture as required are roasted in sun ray for 2 to 3 days.
b) All the roasted and other ingredients of mixture are mixed as per volume/weight;
c) All the mixed ingredient are heated till boiling temperature for a certain time.
d) After heating the extract is cooled to a room temperature.
e) The extract is then processed of purification so that no solid portion and malformation comes out.

9) The process for preparing the novel synergistic herbal formulation as claimed in claim 1 in which above all ingredients are mixed in a ratio such as herein described.

10) The novel synergistic herbal formulation as claimed in claim 1 which can be used externally and neuro pathways.
